(19) United States
(12) Patent Application Publication
(10) Pub. No.: US 2003/0083305 A1
(43) Pub. Date: May 1, 2003

(54) Viral interferon antagonists and uses therefor

(76) Inventors: Peter Palese, Leonia, NJ (US); Adolfo Garcia-Sastre, New York, NY (US)

Correspondence Address: PENNIE AND EDMONDS, 1155 AVENUE OF THE AMERICAS, NEW YORK, NY, 100362711

(21) Appl. No.: 10/269,513

(22) Filed: Oct. 10, 2002

Related U.S. Application Data
(60) Provisional application No. 60/328,573, filed on Oct. 10, 2001.

(51) Int. Cl. A61K 48/00

(52) U.S. Cl. 514/44; 424/93.2

(57) ABSTRACT

The present invention relates to compositions comprising one or more viral interferon antagonists and methods of utilizing said compositions to modulate the cellular interferon immune response. In particular, the present invention relates to pharmaceutical compositions comprising one or more viral interferon antagonists and methods of utilizing said compositions to prevent, treat or ameliorate an immune disorder characterized by aberrant interferon expression and/or activity. The invention also relates to methods of treating, preventing or ameliorating the symptoms of an inflammatory disorder comprising administering to a subject in need thereof one or more viral interferon antagonist. The present invention also relates to compositions comprising fusion proteins comprising one or more viral interferon antagonists and a heterologous polypeptide, and methods of using said compositions to modulate the cellular interferon immune response. The present invention further relates to articles of manufacture comprising one or more viral interferon antagonists or fusion proteins.

### 1. FIELD OF THE INVENTION

The present invention relates to compositions comprising viral interferon antagonists and methods for modulating interferon expression and/or activity in vitro and/or in vivo utilizing said compositions. In particular, the present invention relates to compositions comprising one or more viral interferon antagonists and methods of modulating the immune response in a subject by administering to a subject in need thereof said compositions. The present invention also relates to methods for modulating Th1/Th2 differentiation and/or Th1 replication utilizing compositions comprising one or more viral interferon antagonists. The present invention also relates to methods for preventing, treating or ameliorating symptoms associated with a Th1 or Th1-like related immune disorder in a subject, said methods comprising administering to said subject an effective amount of one or more viral interferon antagonists. The present invention further relates to articles of manufacture comprising viral interferon antagonists at appropriate unit dosages for methods of administration of the viral interferon antagonists to a subject, said viral interferon antagonists being contained in appropriate vessels or containers.

### 2. BACKGROUND OF THE INVENTION

2.1 Interferon and Viral Interferon Antagonists

Interferons are a family of cytokines originally identified by their ability to confer cellular resistance to viral infection and which are also involved in cell growth regulation and immune activation (Garcin et al., 1999, J. Viol. 73(8):6559). There are two types of IFNs, type I interferon or, IFNs α/β (which include IFNα and IFNβ) and type II interferon or IFNY. IFN α/β is usually induced within hours after viral infection. Once it is synthesized, it functions in both an autocrine and paracrine fashion to prevent the replication and spread of viruses. Induction of IFN-α/β upon viral infection requires multiple regulatory factors. These factors are mainly at the transcriptional level, inducing the synthesis of mRNAs from the IFN-α/β genes (Algarte et al., 1999 J. Virol. 73:2694; Schafer et al., 1998, J. Biol. Chem. 273:2714).

The anti-viral state induced by type I interferon results in the induction of interferon stimulated genes products including anti-viral proteins such as double-stranded-RNA-dependent protein kinase (PKR) and 2′-5′-oligoadenylate synthatase (Gale et. al., 1998, Phamacol. Ther. 78:29; Stark et al. 1998, Annu. Rev. Biochem. 67: 227)

Many viruses have evolved different mechanisms to subvert the host IFN response. For example, the V protein of SV5 targets STAT1 for proteasome degradation preventing signaling from both type I and type II IFN receptors (Young et al., 2000, Virology, 269:383; Didock et al., 1999, J. Virol., 73:9928). Another example of virally encoded proteins that are capable of antagonizing the host cell's interferon response is the C protein of Sendai Virus (Garcin et al., 1999, J. Virol. 69(1):499). The herpes simplex virus interferon antagonist counteracts the double stranded RNA activated interferon induced protein kinase (PKR) mediated phosphorylation of translation initiation factor CIF-22, preventing the establishment of an IFN-induced block in protein synthesis (He et al., 1997, Proc. Natl. Acad. Sci. USA:94:843). Examples of other virally encoded proteins that inhibit PKR include: vaccinia virus E3L and K3L (Beattie et al., 1995, J. Virol. 69(1):499; Shors et al., 1997, Virology 239(2):269) and VA RNA₁, of adenovirus (Kitajewski et al., 1986, Cell 45(2): 195).

2.2 The TH-1 Response

T Lymphocytes are effector cells of the immune response. Two distinct types of T lymphocytes are recognized: CD8⁺ cytotoxic T lymphocytes (CTLs) and CD4⁺ helper T lymphocytes (Th cells). Th cells are involved in both humoral and cell-mediated forms of effector immune responses. With respect to the humoral or antibody immune response, antibodies are produced by B lymphocytes through interactions with Th cells. Specifically, extracellular antigens are endocytosed by antigen-presenting cells (APCs), processed, and presented preferentially in association with class II major histocompatibility complex (MHC) molecules to CD4⁺ class II MHC-restricted Th cells. These Th cells in turn activate B lymphocytes, resulting in antibody production.

The cell-mediated or cellular immune response, functions to neutralize microbes which inhabit intracellular locations. Foreign antigens, such as, for example, viral antigens, are synthesized within infected cells and presented on the surfaces of such cells in association with class I MHC molecules. This, then, leads to the stimulation of the CD8⁺ class I MHC-restricted CTLs.

Th cells are composed of at least two distinct subpopulations, termed Th1 and Th2 cell subpopulations. While such subpopulations were originally discovered in murine systems (reviewed in Mosmann, T. R. and Coffman, R. L., 1989, Ann. Rev. Immunol. 7:145), the existence of Th1- and Th2-like subpopulations has also been established in humans (Del Prete, A. F. et al., 1991, J. Clin. Invest. 88:346; Wiernenga, E. A. et al., 1990, J. Imm. 144:4651; Yamamura, M. et al., 1991, Science 254:277; Robinson, D. et al., 1993, J. Allergy Clin. Imm. 92:313).

It has been noted that the ability of the different Th cell types to drive different immune effector responses is due to the exclusive combinations of cytokines which are expressed within a particular Th cell subpopulation. For example, Th1 cells are known to secrete interleukin-2 (IL-2), interferon-γ (IFN-γ), and lymphotoxin, while TH2 cells secrete interleukin-4 (IL-4), interleukin-5 (IL-5), and interleukin-10 (IL-10).

Once Th1 and Th2 subpopulations are expanded, the cell types tend to negatively regulate one another through the actions of cytokines unique to each. For example, Th1-produced IFN-γ negatively regulates Th2 cells, while Th2-produced IL-10 negatively regulates Th1 cells. Moreover, cytokines produced by Th1 and Th2 antagonize the effector functions of one another (Mosmann, T. R. and Moore, 1991, Immunol. Today 12:49).

Further, while Th1-mediated inflammatory responses to many pathogenic microorganisms are beneficial, such responses to self antigens are usually deleterious. It has been suggested that the preferential activation of Th1-like responses is central to the pathogenesis of such human inflammatory autoimmune diseases as multiple sclerosis and insulin-dependent diabetes. For example, Th1-type cytokines predominate in the cerebrospinal fluid of patients with multiple sclerosis, pancreases of insulin-dependent diabetes patients, thyroid glands of Hashimoto's thyroiditis, and gut of Crohn's disease patients, suggesting that such patients mount a Th1-like, not a Th2-like, response to the antigen(s) involved in the etiopathogenesis of such disorders.

Interferon γ plays a role in the differentiation of CD4⁺ cells into Th1 cells and thus plays a role in the generation of a cellular immune response (Stark et al., 1998, Annu. Rev. Biochem. 67:227). Interferon-α plays a role in the production of interferon γ. Exposing CD4+ and CD8+cells to the type I interferon, interferon-α, results in a 10 fold increase in the level of interferon γ produced by these cells (Brinkmann, et. al., 1993, J. Exp. Med. 178(5):1655). Thus, interferon-α plays a role in the differentiation of CD4⁺ T cells into Th1 cells. Antagonizing type I interferon provides a strategy of controlling Th cell differentiation and limiting the Th1 response and thus provides a therapeutic approach to treating Th1 related disorders.

Discussion or citation of a patent, patent publication or other reference herein shall not be construed as an admission that such patent, patent publication or citation is prior art to the present invention.

### 3. SUMMARY OF THE INVENTION

The present invention provides compositions comprising one or more viral interferon antagonists and methods for modulating the immune response utilizing said compositions. The present invention also provides compositions comprising one or more viral interferon antagonists and methods for modulating gene expression, in particular, interferon gene expression, in vitro and/or in vivo utilizing said compositions. In one aspect, the present invention provides methods for down-regulating interferon expression and/or activity and interferon-dependent gene expression in vitro and/or in vivo utilizing compositions comprising one or more viral interferon antagonists. In another aspect, the present invention provides methods for enhancing gene expression in vitro and/or in vivo utilizing compositions comprising one or more viral interferon antagonists. The present invention also provides methods for modulating Th1/Th2 differentiation and/or Th1 replication in vitro and/or in vivo utilizing compositions comprising one or more viral interferon antagonists. The present invention further provides methods for interfering with interferon-mediated enhancement of antibody production.

Viral interferon antagonists are viral proteins, polypeptides, derivatives, analogs or fragments thereof that impair the cellular interferon immune response. In particular, a viral interferon antagonist reduces or inhibits interferon expression and/or activity in vitro and/or in vivo. Examples of viral interferon antagonists include, but are not limited to, influenza virus NS1, respiratory syncytial virus (RSV) NS2, and Ebola virus VP35. In a preferred embodiment, at least one of the viral interferon antagonists utilized in the compositions and methods of the invention is influenza virus NS1, more particularly influenza A virus NS1.

The present invention provides compositions comprising one or more viral interferon antagonists or nucleotide sequences encoding one or more viral interferon antagonists, and a carrier. The present invention also provides pharmaceutical compositions comprising one or more viral interferon antagonists or nucleotide sequences encoding one or more viral interferon antagonists, and a pharmaceutically acceptable carrier. In one embodiment of the invention, a pharmaceutical composition comprises one or more viral interferon antagonists in an amount effective to reduce or inhibit interferon expression and/or activity in vitro and/or in vivo, and a pharmaceutically acceptable carrier. In a preferred embodiment, a pharmaceutical composition comprises one or more viral interferon antagonists in an amount effective to reduce or inhibit interferon expression and/or activity in vivo, and a pharmaceutically acceptable carrier. In another embodiment of the invention, a pharmaceutical composition comprises one or more viral interferon antagonists in an amount effective to enhance gene expression in vitro and/or in vivo, and a pharmaceutically acceptable carrier. In another embodiment of the invention, a pharmaceutical composition comprises one or more viral interferon antagonists in an amount effective to reduce or inhibit Th1/Th2 cell or Th1/Th2-like cell differentiation and/or Th1 or Th1-like replication in vitro and/or in vivo, and a pharmaceutically acceptable carrier. In a preferred embodiment, a pharmaceutical composition comprises one or more viral interferon antagonists in an amount effective to reduce or inhibit Th1/Th2 cell or Th1/Th2-like cell differentiation and/or Th1 replication in vivo.

In another embodiment, a pharmaceutical composition comprises one or more viral interferon antagonists in an amount effective to prevent, treat, or ameliorate one or more symptoms associated with a Th1 or Th1-like related disorder, and a pharmaceutically acceptable carrier. In a preferred embodiment, a pharmaceutical composition comprises one or more viral interferon antagonists in an amount effective to prevent, treat, or ameliorate one or more symptoms associated with an inflammatory disease or disorder. Preferably, the pharmaceutical compositions of the invention are sterile and in a form suitable for administration to a subject, preferably an animal subject, more preferably a mammalian subject, and most preferably a human subject.

The present invention provides methods of modulating an immune response in a subject by administering to said subject a composition comprising one or more viral interferon antagonists, or nucleotide sequences encoding one or more viral interferon antagonists. In particular, the present invention provides methods of modulating an interferon immune response in a subject comprising administering to said subject a prophylactically or therapeutically effective amount of one or more viral interferon antagonists. The present invention also provides methods of preventing, treating, or ameliorating one or more symptoms associated with an immune disorder, in particular, an immune disorder characterized by aberrant interferon expression and/or activity in a subject comprising administering to said subject a prophylactically or therapeutically effective amount of one or more viral interferon antagonists. In particular, the present invention provides methods of preventing, treating, or ameliorating one or more symptoms associated with a Th1 or Th1-like related disorder in a subject comprising administering to said subject a prophylactically or therapeutically effective amount of one or more viral interferon antagonists. Examples of Th1 or Th1-like related disorders include, but are not limited to, chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease and sarcoidosis. In a specific embodiment, the present invention provides methods of preventing, treating or ameliorating one or more symptoms associated with an inflammatory disease or disorder in subject comprising administering to said subject a prophylactically or therapeutically effective amount of one or more viral interferon antagonists.

The present invention provides fusion proteins (a.k.a. VIA fusion proteins) comprising a viral interferon antagonist and a heterologous polypeptide. The present invention also provides compositions comprising one or more VIA fusion proteins or nucleotide sequences encoding one or more VIA fusion proteins and methods for utilizing said compositions. The fusion proteins of the invention can be utilized to, e.g., modulate gene expression, modulate Th1/Th2 differentiation and/or Th1 replication, and prevent, treat or ameliorate one or more symptoms associated with an immune disorder characterized by aberrant interferon expression and/or activity. In particular, the fusion proteins of the invention can be utilized to prevent, treat or ameliorate one or more symptoms associated with a Th1 or Th1-like related disorder. In a specific embodiment, the fusion proteins of the invention are utilized to prevent, treat or ameliorate one or more symptoms associated with an inflammatory disease or disorder. The VIA fusion proteins can be used alone or in combination with viral interferon antagonists in the compositions and methods described herein.

The present invention also encompasses the use of viral interferon antagonists or VIA fusion proteins in combinatorial therapies for the prevention, treatment and amelioration of one or more symptoms associated with immune disorders. In particular, the present invention provides methods for preventing, treating or ameliorating one or more symptoms associated with an immune disorder characterized by aberrant IFN expression and/or activity in a subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. The present invention provides methods for preventing, treating or ameliorating one or more symptoms associated with a Th1 or Th1-like related disorder in subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. In a specific embodiment, the present invention provides methods of preventing, treating or ameliorating one or more symptoms of an inflammatory disorder in subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. The present invention encompasses the use of one or more viral interferon antagonists or VIA fusion proteins in cycling therapy for the treatment, prevention, or amelioration of one or more symptoms of an immune disorder. Preferably, the combinatorial therapies of the present invention have an additive or synergistic effect while reducing or avoiding unwanted or adverse side effects.

The present invention also provides articles of manufacture comprising viral interferon antagonists or VIA fusion proteins at appropriate unit dosages for the method of administration of the viral interferon antagonists or VIA fusion proteins to a subject, said viral interferon antagonists or VIA fusion proteins being contained in appropriate vessels or containers. Preferably, the articles of manufacture further comprise instructions for use of the viral interferon antagonists.

3.1. Definitions

Analog: As used herein, the term “analog” refers to a polypeptide that possesses a similar or identical function as a viral protein or polypeptide with interferon antagonist activity but does not necessarily comprise a similar or identical amino acid sequence of the viral protein or polypeptide, or possess a similar or identical structure of the viral protein or polypeptide. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a viral protein or polypeptide; (b) a polypeptide encoded by a nucleotide sequence that hybridizes under stringent conditions to a nucleotide sequence encoding a viral protein or polypeptide of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 35 contiguous amino acid residues, at least 40 contiguous amino acid residues, at least 50 contiguous amino acid residues, at least 55 contiguous amino acid residues, at least 60 contiguous amino acid residues, at least 65 contiguous amino acid residues, at least 70 contiguous amino acid residues, at least 75 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a viral protein or polypeptide. A polypeptide with similar structure to a viral protein or polypeptide refers to polypeptide that has a similar secondary, tertiary, or quaternary structure of a viral protein or polypeptide. The structure of a polypeptide can be determined by methods known to those skilled in the art, including, but not limited to, peptide sequencing, X-ray crystallography, nuclear magnetic resonance, circular dichroism, and crystallographic electron microscopy.

Hybridized under stringent conditions: As used herein the term “hybridizes under stringent conditions” describes conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6×sodium chloride/sodium citrate (SSC) at about 45° C., followed by one or more washes in 0.1×SSC, 0.2% SDS at about 68° C. A preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides. In a specific embodiment, a nucleotide sequence encoding a polypeptide hybridizes over its full length to a nucleotide sequence encoding a viral protein or polypeptide having interferon antagonist activity, preferably, said polypeptide that hybridizes to the viral protein or polypeptide has interferon antagonist activity.

Derivative: As used herein, the term “derivative” refers to a polypeptide that comprises an amino acid sequence of a viral protein or polypeptide, such as a viral interferon antagonist, which has been altered by the introduction of amino acid residue substitutions, deletions or additions, or by the covalent attachment of any type of molecule to the polypeptide. The term “derivative” as used herein also refers to a viral protein or polypeptide which has been modified, e.g., by the covalent attachment of any type of molecule to the viral protein or polypeptide. For example, but not by way of limitation, a viral protein or polypeptide may be modified, e.g., by proteolytic cleavage, linkage to a cellular ligand or other protein, etc. A derivative of a viral protein or polypeptide may be modified by chemical modifications using techniques known to those of skill in the art (e.g., by acylation, phosphorylation, carboxylation, glycosylation, selenium modification and sulfation). Further, a derivative of a viral protein or polypeptide may contain one or more non-classical amino acids. A polypeptide derivative possesses a similar or identical function as a viral protein or polypeptide. In a preferred embodiment, a polypeptide derivative retains the interferon antagonist activity of a viral protein or polypeptide.

Fragment: As used herein, the term “fragment” refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a viral interferon antagonist. In a preferred embodiment, a fragment of a viral interferon antagonist has interferon antagonist activity.

Functional fragment: As used herein, the term “functional fragment” refers to a fragment of a viral interferon antagonist which has interferon antagonist activity.

Fusion protein: As used herein, the term “fusion protein” or “viral interferon antagonist (“VIA”) fusion protein” refers to a polypeptide that comprises an amino acid sequence of a viral interferon antagonist, and an amino acid sequence of a heterologous polypeptide (i.e., an unrelated protein such as, e.g., a different viral interferon antagonist, or a viral protein or polypeptide lacking interferon antagonist activity). In a preferred embodiment, a fusion protein or VIA fusion protein has interferon antagonist activity. In another preferred embodiment, a fusion protein comprises a viral interferon antagonist, or functional fragment or derivative thereof and a heterologous polypeptide that targets the viral interferon antagonist, functional fragment or derivative thereof to a particular site in a subject.

Immune disorder characterized by aberrant IFN expression and/or activity: As used herein, the phrase “immune disorder characterized by aberrant IFN expression and/or activity” refers to an immune disorder in which IFN expression and/or activity contributes to the severity or duration of the immune disorder, or one or more symptoms associated with the immune disorder.

Interferon Antagonist Activity: As used herein the phrase “interferon antagonist activity” refers to a viral protein or polypeptide, or fragment, derivative, or analog thereof that reduces or inhibits the cellular interferon immune response. In particular, a viral protein or polypeptide, or fragment, derivative, or analog thereof that has interferon antagonist activity reduces or inhibits interferon expression and/or activity. A viral protein or polypeptide with interferon antagonist activity may preferentially affect the expression and/or activity of one type of interferon (IFN).

Isolated: An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. Moreover, an “isolated” nucleic acid molecule can be substantially free of other cellular material, or culture medium when produced by recombinant techniques, or substantially free of chemical precursors or other chemicals when chemically synthesized.

An “isolated” polypeptide is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Nucleic Acids: As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA. In one embodiment, the nucleotide sequences comprise a contiguous open reading frame encoding a viral interferon antagonist, e.g., a cDNA molecule.

Prevent: As used herein, the terms “prevent”, “preventing”, and “prevention” refer to the prevention of the onset or recurrence of one or more symptoms of an immune disorder characterized by aberrant interferon expression and/or activity, a Th1 or Th1-like related disorder, or an inflammatory disorder in subject as a result of the administration of one or more viral interferon antagonists or VIA fusion proteins, or a composition comprising one or more viral interferon antagonists or VIA fusion proteins.

Prophylactically effective amount: As used herein, the term “prophylactically effective amount” refers to the amount of one or more viral interferon antagonists or VIA fusion proteins, or the amount of a composition comprising one or more viral interferon antagonists or VIA fusion proteins sufficient to: prevent the onset or recurrence of one or more symptoms associated with an immune disorder characterized by aberrant interferon expression and/or activity; prevent the onset or recurrence of one or more symptoms associated with a Th1 or Th1-like related disorder; prevent the onset or recurrence of one or more symptoms associated with an inflammatory disorder; reduce or inhibit the expression of IFN, in particular IFN-γ expression, as assessed by in vitro and/or in vivo assays; reduce or inhibit IFN activity as assessed by in vitro and/or in vivo assays; or to reduce or inhibit Th1/Th2 differentiation and/or Th1 replication as assessed by in vitro and/or in vivo assays.

In a preferred embodiment, the term “prophylactically effective amount” as used herein refers to the amount of one or more viral interferon antagonists or VIA fusion proteins, or the amount of a composition comprising one or more viral interferon antagonists or VIA fusion proteins sufficient to: prevent the onset or recurrence of one or more symptoms associated with an immune disorder characterized by aberrant interferon expression and/or activity; prevent the onset or recurrence of one or more symptoms associated with a Th1 or Th1-like related disorder; prevent the onset or recurrence of one or more symptoms associated with an inflammatory disorder; reduce or inhibit the expression of IFN, in particular IFN-γ expression, in vivo as assessed by in vitro and/or in vivo assays; reduce or inhibit IFN activity in vivo as assessed by in vitro and/or in vivo assays; or to reduce or inhibit Th1/Th2 differentiation and/or Th1 replication in vivo as assessed by in vitro and/or in vivo assays.

Subject: As used herein, the term “subject” in the context of individuals treated for a disorder refers to an animal subject, more preferably a mammalian subject, and most preferably a human subject.

Therapeutically effective amount: As used herein, the term “therapeutically effective amount” refers to the amount of one or more viral interferon antagonists or VIA fusion proteins, or the amount of a composition comprising one or more viral interferon antagonists or VIA fusion proteins sufficient to: reduce the severity or duration of an immune disorder characterized by aberrant interferon expression and/or activity; reduce the duration of a disease course, ameliorate one or more symptoms associated with an immune disorder characterized by aberrant interferon expression and/or activity; reduce or inhibit the severity or duration of a Th1 or Th1-like related disorder; reduce or inhibit one or more symptoms associated with a Th1 or Th1-like related disorder; reduce or inhibit the severity or duration of an inflammatory disease or disorder; reduce or inhibit one or more symptoms associated with an inflammatory disease or disorder; reduce or inhibit the expression of IFN as assessed by in vitro and/or in vivo assays; reduce or inhibit IFN activity as assessed by in vitro and/or in vivo assays; or reduce or inhibit Th1/Th2 differentiation and/or Th1 replication as assessed by in vitro and/or in vivo assays.

In a preferred embodiment, the term “therapeutically effective amount” as used herein refers to the amount of one or more viral interferon antagonists or VIA fusion proteins, or the amount of a composition comprising one or more viral interferon antagonists or VIA fusion proteins sufficient to: reduce the severity or duration of an immune disorder characterized by aberrant interferon expression and/or activity; reduce the duration of a disease course, ameliorate one or more symptoms associated with an immune disorder characterized by aberrant interferon expression and/or activity; reduce or inhibit the severity or duration of a Th1 or Th1-like related disorder; reduce or inhibit one or more symptoms associated with a Th1 or Th1-like related disorder; reduce or inhibit the severity or duration of an inflammatory disease or disorder; reduce or inhibit one or more symptoms associated with an inflammatory disease or disorder; reduce or inhibit the expression of IFN in vivo as assessed by in vitro and/or in vivo assays; reduce or inhibit IFN activity in vivo as assessed by in vitro and/or in vivo assays; or reduce or inhibit Th1/Th2 differentiation and/or Th1 replication in vivo as assessed by in vitro and/or in vivo assays.

Treat: As used herein, the terms “treat”, “treatment”, and “treating” refer to: the reduction in the severity of an immune disorder characterized by aberrant interferon expression and/or activity, a Th1 or Th1-like related disorder, or an inflammatory disorder; the reduction in the duration of a disease course of an immune disorder characterized by aberrant interferon expression and/or activity, a Th1 or Th1-like related disorder, or an inflammatory disorder; the amelioration of one or more symptoms associated with an immune disorder characterized by aberrant interferon expression and/or activity, a Th1 or Th1-like related disorder, or an inflammatory disorder; the reduction or inhibition of IFN expression, in particular IFN-γ expression, as assessed by in vitro and/or in vivo assays; the reduction or inhibition of IFN activity as assessed by in vitro and/or in vivo assays; or the reduction or inhibition of Th1/Th2 differentiation and/or Th1 Th1 replication as assessed by in vitro and/or in vivo assays. In a specific embodiment, the administration of one or more viral interferon antagonists to a subject results in one or more beneficial effects for the subject, but does not result in a cure of the disorder.

### 4. BRIEF DESCRIPTION OF THE FIGURES

FIG. 1. Expression of Ebola virus VP35 protein inhibits dsRNA- or virus-mediated induction of an ISRE. FIG. 1A. Fold induction of an ISRE promoter-CAT reporter gene in the presence of empty vector, NS1 expression plasmid, or VP35 expression plasmid. The CAT activities were normalized to the corresponding luciferase activities to determine fold induction. FIG. 1B. Western blot showing NS1, VP35, and Ebola virus NP expression. 293 cells were transfected with 4 μg of the indicated plasmids. Forty-eight hours later, cell lysates were prepared and Western blots were performed by using the indicated antiserum.

FIG. 2. The VP35 protein of Ebola virus inhibits induction of the IFN-β promoter. FIG. 2A. Inhibition of induction of the mouse IFN-β promoter. 293 cells were transfected with 4 μg of the indicated expression plasmid plus 0.3 μg each of the reporter plasmids pIFN-β-CAT and pGL2-Control. Twenty-four hours posttransfection, the cells were mock-transfected or transfected with 40 μg of polyl:polyC. FIG. 2B. Northern blot showing VP35-mediated inhibition of endogenous IFN-induction. 293 cells were transfected with either empty vector or VP35 expression plasmid. Twenty-four hours later, the cells were mock-infected or infected with influenza delNS 1 virus (delNS 1) or Sendai virus (SeV) (moi=1). Total RNA was prepared from cells at ten or twenty hours posttransfection. Mock-transfected cell RNA was prepared at the same time as the twenty hour postinfection samples. Northern blots were performed to detect IFN-β or β-actin mRNAs. Note that less total RNA was obtained when cells, including the mock-infected cells, were lysed at the twenty hour postinfection time point.

FIG. 3. The Ebola virus VP35 protein inhibits type I IFN induction when coexpressed with Ebola virus NP. Fold induction of the IFN-inducible ISRE-driven reporter in the presence of empty vector, VP35, NP, or VP35 plus NP. 293 cells were transfected with a total of 4 μg of expression plasmid, including 2 μg of a plasmid encoding an individual protein and 2 μg of a second plasmid (either empty vector or a second expression plasmid) plus 0.3 μg each of the reporter plasmids pHISG-54-CAT and pGL2-Control. Twenty-four hours posttransfection, the cells were mock-treated or treated with the indicated IFN inducer. Twenty-four hours postinduction, CAT and luciferase assays were performed. The CAT activities were normalized to the corresponding luciferase activities to determine fold induction.

FIG. 4. Stimulation of luciferase expression from pGL2-Control by co-expression with a viral interferon antagonist. Transfection of an interferon antagonist can enhance expression of other genes. The ability to enhance expression of transfected genes may be useful when maximal gene expression is desired.

### 5. DETAILED DESCRIPTION OF THE INVENTION

The present invention provides compositions comprising one or more viral interferon antagonists and methods for modulating gene expression, in particular, interferon gene expression, in vitro and/or in vivo utilizing said compositions. In one aspect, the present invention provides methods for down-regulating interferon expression and/or activity and interferon-dependent gene expression in vitro and/or in vivo utilizing compositions comprising one or more viral interferon antagonists. In another aspect, the present invention provides methods for enhancing gene expression in vitro and/or in vivo utilizing compositions comprising one or more viral interferon antagonists. The present invention also provides methods for modulating Th1/Th2 differentiation and/or Th1 replication in vitro and/or in vivo utilizing compositions comprising one or more viral interferon antagonists. The present invention further provides methods for interfering with interferon-mediated enhancement of antibody production.

The present invention provides compositions comprising one or more viral interferon antagonists, and a carrier. The present invention also provides compositions comprising nucleotide sequences encoding one or more viral interferon antagonists, and a carrier. The present invention also provides pharmaceutical compositions comprising one or more viral interferon antagonists, and a pharmaceutically acceptable carrier. The present invention further provides pharmaceutical compositions comprising nucleotide sequences encoding one or more viral interferon antagonists, and a pharmaceutically acceptable carrier.

The invention provides methods of modulating the cellular interferon immune response in a subject by administering to said subject a composition comprising one or more viral interferon antagonists or nucleotide sequences encoding one or more viral interferon antagonists. In particular, the present invention provides methods of modulating an interferon immune response in a subject comprising administering to said subject a prophylactically or therapeutically effective amount of one or more viral interferon antagonists, or nucleotide sequences encoding one or more viral interferon antagonists. The present invention also provides methods of preventing, treating, or ameliorating one or more symptoms associated with an immune disorder characterized by aberrant interferon expression and/or activity in a subject comprising administering to said subject a prophylactically or therapeutically effective amount of one or more viral interferon antagonists, or nucleotide sequences encoding one or more viral interferon antagonists. In particular, the present invention provides methods of preventing, treating, or ameliorating one or more symptoms associated with a Th1 or Th1-like related disorder in a subject comprising administering to said subject a prophylactically or therapeutically effective amount of one or more viral interferon antagonists, or nucleotide sequences encoding one or more viral interferon antagonists. Examples of Th1 or Th1-like related disorders include, but are not limited to, chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease and sarcoidosis. In a specific embodiment, the present invention provides methods of preventing, treating or ameliorating one or more symptoms associated with an inflammatory disease or disorder in subject comprising administering to said subject a prophylactically or therapeutically effective amount of one or more viral interferon antagonists, or nucleotide sequences encoding one or more viral interferon antagonists.

The present invention provides fusion proteins (a.k.a. VIA fusion proteins) comprising a viral interferon antagonist and a heterologous polypeptide. The present invention also provides compositions comprising one or more VIA fusion proteins and methods for utilizing said compositions. The fusion proteins of the invention can be utilized to, e.g., modulate gene expression, modulate Th1/Th2 differentiation and/or Th1 replication, and prevent, treat or ameliorate one or more symptoms associated with an immune disorder characterized by aberrant interferon expression and/or activity. In particular, the fusion proteins of the invention can be utilized to prevent, treat or ameliorate one or more symptoms associated with a Th1 or Th1-like related disorder. In a specific embodiment, the fusion proteins of the invention are utilized to prevent, treat or ameliorate one or more symptoms associated with an inflammatory disease or disorder. The VIA fusion proteins can be used alone or in combination with viral interferon antagonists in the compositions and methods described herein.

The present invention also encompasses the use of viral interferon antagonists or VIA fusion proteins in combinatorial therapies for the prevention, treatment and amelioration of one or more symptoms associated with immune disorders. In particular, the present invention provides methods for preventing, treating or ameliorating one or more symptoms associated with an immune disorder characterized by aberrant IFN expression and/or activity in a subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. The present invention provides methods for preventing, treating or ameliorating one or more symptoms associated with a Th1 or Th1-like related disorder in subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. In a specific embodiment, the present invention provides methods of preventing, treating or ameliorating one or more symptoms of an inflammatory disorder in subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. The present invention encompasses the use of one or more viral interferon antagonists or VIA fusion proteins in cycling therapy for the treatment, prevention, or amelioration of one or more symptoms of an immune disorder. Preferably, the combinatorial therapies of the present invention have an additive or synergistic effect while reducing or avoiding unwanted or adverse side effects.

The present invention also provides articles of manufacture comprising viral interferon antagonists or VIA fusion proteins at appropriate unit dosages for the method of administration of the viral interferon antagonists to a subject, said viral interferon antagonists being contained in appropriate vessels or containers. Preferably, the articles of manufacture further comprise instructions for use of the viral interferon antagonists or VIA fusion proteins.

5.1. Viral Interferon Antagonists

The present invention provides compositions comprising one or more viral interferon antagonists and methods of utilizing one or more viral interferon antagonists. Viral interferon antagonists are viral proteins, polypeptides, derivatives, analogs or fragments thereof that impair the cellular interferon immune response. In particular, viral interferon antagonists reduce or inhibit interferon expression and/or activity in vitro and/or in vivo. Preferably, viral interferon antagonists reduce or inhibit interferon expression and/or activity in vivo.

In a specific embodiment, a viral interferon antagonist reduces cellular interferon expression by approximately 5%, approximately 10%, approximately 15%, approximately 20%, approximately 25%, approximately 30%, approximately 35%, approximately 40%, approximately 45%, approximately 50%, approximately 55%, approximately 60%, approximately 65%, approximately 70%, approximately 75%, approximately 80%, approximately 85%, approximately 90%, approximately 95%, or approximately 98% relative to cellular interferon expression in the absence of the viral interferon antagonist as determined by an in vitro assay (e.g., an immunoassay such as an ELISA) or in vivo assay (e.g., in situ hybridization assay) well known to one of skill in the art or described herein. In another specific embodiment, a viral interferon antagonist reduces cellular interferon activity by approximately 5%, approximately 10%, approximately 15%, approximately 20%, approximately 25%, approximately 30%, approximately 35%, approximately 40%, approximately 45%, approximately 50%, approximately 55%, approximately 60%, approximately 65%, approximately 70%, approximately 75%, approximately 80%, approximately 85%, approximately 90%, approximately 95%, or approximately 98% relative to cellular interferon activity in the absence of the viral interferon antagonist as determined by an in vitro (e.g., an EMSA) or in vivo assay well known to one of skill in the art or described herein. In preferred embodiment, a viral interferon antagonist reduces cellular interferon expression by approximately 5%, approximately 10%, approximately 15%, approximately 20%, approximately 25%, approximately 30%, approximately 35%, approximately 40%, approximately 45%, approximately 50%, approximately 55%, approximately 60%, approximately 65%, approximately 70%, approximately 75%, approximately 80%, approximately 85%, approximately 90%, approximately 95%, or approximately 98% relative to cellular interferon expression in the absence of the viral interferon antagonist and reduces cellular interferon activity by approximately 5%, approximately 10%, approximately 15%, approximately 20%, approximately 25%, approximately 30%, approximately 35%, approximately 40%, approximately 45%, approximately 50%, approximately 55%, approximately 60%, approximately 65%, approximately 70%, approximately 75%, approximately 80%, approximately 85%, approximately 90%, approximately 95%, or approximately 98% relative to cellular interferon activity in the absence of the viral interferon antagonist as determined by an in vitro or in vivo assay well known to one of skill in the art or described herein.

Viral interferon antagonists can be derived from any virus, including, but not limited to, RNA viruses including paramyxoviruses (e.g., Sendai virus, parainfluenza virus, mumps, and Newcastle disease virus), morbilliviruses (e.g., measles virus, canine distemper virus and rinderpest virus), pneumoviruses (e.g., respiratory syncytial virus and bovine respiratory virus), rhabdoviruses (e.g., vesicular stomatitis virus and lyssavirus), hepatitis C viruses, orthomyxoviruses (e.g., influenza virus), bunyaviruses, hantaviruses, ebola viruses and retroviruses (e.g., HTLV and HIV), and DNA viruses, including vaccinia, adenoviruses, hepadna viruses, herpes viruses and poxviruses. Examples of viral interferon antagonists include, but are not limited to, influenza virus NS1, respiratory syncytial virus (RSV) NS2, vaccinia virus E3L, and Ebola virus VP35. In certain embodiments, at least one of the viral interferon antagonists utilized in the compositions and methods of the invention is influenza virus NS1, more particularly influenza A virus NS1. In certain other embodiments, influenza virus NS1, in particular influenza A virus NS1, is not utilized in the compositions and methods of the invention.

The nucleotide sequence encoding a viral interferon antagonist may be obtained from any information available to one of skill in the art (i.e., from GenBank, the literature, or by routine cloning). For example, the nucleotide sequence encoding influenza virus NS1, Ebola virus VP35, and RSV NS2 can be obtained from GenBank Accession Nos. Z26866, NC_{—}002549, and NC_{—}001781, respectively. If a clone containing a nucleic acid encoding a particular viral interferon antagonist is not available, but the sequence of the viral interferon antagonist is known, a nucleic acid encoding the viral interferon antagonist may be chemically synthesized or obtained from a suitable source (e.g., a cDNA library, or nucleic acid, preferably poly A+RNA, isolated from any tissue or cells expressing the viral interferon antagonist) by PCR amplification using synthetic primers hybridizable to the 3′ and 5′ ends of the sequence or by cloning using an oligonucleotide probe specific for the particular nucleotide sequence. Amplified nucleic acids generated by PCR may then be cloned into replicable cloning vectors using any method well known in the art.

5.1.1. Methods of Identifying Viral Interferon Antagonists

Any viral protein or polypeptide may be assayed for interferon antagonist activity. The viral protein or polypeptide to be tested for interferon antagonist activity can be obtained from any virus. These proteins include, for example, NS1 and other analogous proteins originating from various types of viruses. Such viruses may include, but are not limited to RNA viruses including paramyxoviruses (e.g., Sendai virus, parainfluenza virus, mumps, and Newcastle disease virus), morbilliviruses (e.g., measles virus, canine distemper virus and rinderpest virus), pneumoviruses (e.g., respiratory syncytial virus and bovine respiratory virus), rhabdoviruses (e.g., vesicular stomatitis virus and lyssavirus), hepatitis C viruses, orthomyxoviruses (e.g., influenza virus), bunyaviruses, hantaviruses, ebola viruses and retroviruses (e.g., HTLV and HIV), and DNA viruses, including vaccinia, adenoviruses, hepadna viruses, herpes viruses and poxviruses.

The viral proteins or polypeptides to be tested for interferon antagonist activity may be provided to the assay system to be used as an isolated protein or fragment thereof. Alternatively, nucleic acids encoding the viral proteins, polypeptides or fragments thereof may be provided to the assay system.

Viral proteins or polypeptides to be tested for interferon antagonist activity may be isolated or purified from a virus or viral extract using standard techniques known to those of skill in the art. The viral protein or polypeptide to be tested may be expressed recombinantly using standard techniques known to those of skill in the art. Nucleic acids encoding viral proteins or polypeptides to be tested for interferon antagonist activity may be supplied using standard techniques known to those of skill in the art. Nucleic acids encoding viral proteins to be tested should be operatively linked to the appropriate regulatory elements to allow for their expression. Such nucleic acids may be supplied by way of plasmid, viral vector, bacteriophage etc. and may be operatively linked to regulatory elements selected from viral promoter elements, inducible promoters, constitutive promoters etc. Viral proteins or polypeptides with interferon antagonizing activity can be identified utilizing in vitro and/or in vivo approaches known to one skilled in the art. For example, interferon antagonist activities may be determined by the ability of a viral protein or polypeptide to inhibit or reduce any known interferon based activity, as compared to the absence of the viral protein. Interferon based activities which may be assayed include, but are not limited to, the regulation of interferon regulated promoter elements and genes, the regulation of reporter genes, the increase in translation of proteins, and the regulation of signal transduction pathways, such as the phosphorylation of Janus kinases (JAKS) and signal transducer activators transcription (STATS). Viral proteins or polypeptides with interferon antagonizing activity can also be identified utilizing complementation assays. Such an assay comprises determining the ability of a viral protein or polypeptide to complement the growth and replication of a virus with impaired interferon antagonist activity such as delNS1. Methods for identifying viral proteins or polypeptides with interferon antagonizing activity are described in International Publication No. WO 99/64068 and International Application No. PCT/US01/11543, in particular such assays are described in section 5.1 of International Application No. PCT/US01/11543; the contents International Publication No. PCT/US01/11543 and International Application No. WO 99/64068 are incorporated herein by reference in their entirety.

5.1.2. Derivatives and Analogs of Viral Proteins with Interferon Antagonist Activity

The present invention encompasses the fragments, derivatives, and analogs of viral proteins or polypeptides which have interferon antagonist activity. An isolated nucleic acid molecule encoding a derivative or analog of a viral protein or polypeptide can be created by introducing one or more nucleotide substitutions, additions or deletions into the nucleotide sequence encoding the viral protein or polypeptide such that one or more amino acid substitutions, additions or deletions are introduced into the encoded protein or polypeptide. Mutations can be introduced by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain or antagonize activity. In a preferred embodiment, mutations are introduced into a viral protein or polypeptide with interferon antagonist activity which reduce the immunogenicity of the viral protein or polypeptide. Following mutagenesis, the encoded protein can be expressed recombinantly and the activity of the protein can be determined.

Nucleotide substitutions leading to amino acid substitutions at “non-essential” amino acid residues can be introduced. A “non-essential” amino acid residue is a residue that can be altered from the wild-type sequence without altering the interferon antagonist activity, whereas an “essential” amino acid residue is required for interferon antagonist activity. For example, amino acid residues that are not conserved or only semi-conserved among homologs of various species can be non-essential for activity and thus would be likely targets for alteration. In a preferred embodiment, amino acid substitutions at non-essential amino acid residues are introduced into viral proteins or polypeptides with interferon antagonist activity. Methods of determining the interferon antagonist activity of a derivative or analog of a viral interferon antagonist are described in Section 5.7 below and include assays to assess interferon expression (e.g., northern blot analysis, RT-PCR, and immunoassays) and assays to assess interferon activity (e.g., JAK/STAT activitation by kinase assays or EMSAs).

Derivatives or analogs of a viral protein or polypeptide with interferon antagonist activity include, but are not limited to those molecules comprising regions that are substantially homologous to the viral protein or polypeptide (e.g., in various embodiments, at least 60%, at least 70%, at least 75%, at least 80%, at least 85%, at least 90%, at least 95%, or at least 98% identity over an amino acid sequence of identical size without any insertions or deletions when compared to an aligned sequence in which the alignment is done by a computer homology program known to one of skill in the art) or whose nucleotide sequence is capable of hybridizing under stringent conditions. The determination of percent identity of two amino acid sequences can be determined by any method known to one skilled in the art, including BLAST protein searches.

5.2. Fusion Proteins

The present invention encompasses fusion proteins comprising a viral interferon antagonist and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). Such fusion proteins can be generated by recombinantly fusing or chemically conjugating (e.g., by covalent or non-covalent conjugations) a viral interferon antagonist to a heterologous polypeptide. The fusion can be direct, but may occur through linker sequences. The heterologous polypeptide may be fused to the N-terminus or C-terminus of a viral interferon antagonist.

In one embodiment, a fusion protein comprises a viral interferon antagonist fused to a heterologous signal sequence at its N-terminus. Various signal sequences are commercially available. Eukaryotic heterologous signal sequences include, but art not limited to, the secretory sequences of melittin and human placental alkaline phosphatase (Stratagene; La Jolla, Calif.). Useful prokaryotic heterologous signal sequences include, but are not limited to, the phoA secretory signal (Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989) and the protein A secretory signal (Pharmacia Biotech; Piscataway, N.J.).

In another embodiment, a viral interferon antagonist can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced polypeptides of the invention.

In a preferred embodiment, an affinity label is fused at its amino terminal to the carboxyl terminal of the viral interferon antagonist. The precise site at which the fusion is made in the carboxyl terminal is not critical. The optimal site can be determined by routine experimentation. In another embodiment, an affinity label is fused at its carboxyl terminal to the amino terminal of the viral interferon antagonist.

A variety of affinity labels known in the art may be used, such as, but not limited to, the immunoglobulin constant regions, (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to a viral interferon antagonist, e.g., fragments of green fluorescent protein and the like. Other affinity labels are recognized by specific binding partners and thus facilitate isolation by affinity binding to the binding partner which can be immobilized onto a solid support. Some affinity labels may afford the viral interferon antagonist novel structural properties, such as the ability to form multimers. Dimerization of a viral interferon antagonist with a bound peptide may increase avidity of interaction between the viral interferon antagonist and its partner in the course of antagonizing interferon. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

The affinity labels can also be used to target the viral interferon antagonist to interferon producing cells. Any protein or peptide which binds to a receptor or other protein expressed on a cell that produces interferon can be fused to the viral interferon antagonist in order to target the antagonist to an appropriate cell (i.e a cell that is producing interferon). The viral interferon antagonist can be targeted to the site where interferon acts by fusing it to the binding domain that contacts any cell surface receptor which is internalized into the cell.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CHI of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the α2M polypeptide is intended for in vivo use for humans. Many DNA encoding immunoglobulin light or heavy chain constant regions are known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. USA., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the viral interferon antagonist-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the viral interferon antagonist containing the affinity label. In many instances, there is no need to develop specific antibodies to the viral interferon antagonist.

In a specific embodiment, a fusion protein comprises a viral interferon antagonist fused to the Fc domain of an immunoglobulin molecule or a fragment thereof. In another embodiment, a fusion protein comprises a viral interferon antagonist fused to the CH2 and/or CH3 region of the Fe domain of an immunoglobulin molecule. In another embodiment, a fusion protein comprises a viral interferon antagonist fused to the CH2, CH3, and hinge regions of the Fc domain of an immunoglobulin molecule (see Bowen et al.,1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of the viral interferon antagonist from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting viral interferon antagonist expression in bacterial cells include, but are not limited to, the leader sequences of the Ecoli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (Maclntyre et al., 1990, Mol. Gen. Genet. 221:466-74; Kaiser et al., 1987, Science, 235:312-317).

In a specific embodiment, a fusion protein comprises a viral interferon antagonist and a cell permeable peptide, which facilitates the transport of a protein or polypeptide across the plasma membrane. Examples of cell permeable peptides include, but are not limited to, peptides derived from hepatitis B virus surface antigens (e.g., the PreS2-domain of hepatitis B virus surface antigens), herpes simplex virus VP22, antennapaedia, 6H, 6K, and 6R. See, e.g., Oess et al., 2000, Gene Ther. 7:750-758, DeRossi et al., 1998, Trends Cell Biol 8(2):84-7, and Hawiger, 1997, J. Curr Opin Immunol 9(2): 189-94 for discussion regarding cell permeable peptides.

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific promoter. In a specific embodiment, the expression of a fusion protein is regulated by an inducible promoter.

The invention also provides fusion proteins, which can facilitate solubility and/or expression, and can increase the in vivo half-life of the viral interferon antagonist. Examples include, but are not limited to soluble Ig-tailed fusion proteins. Methods for engineering such soluble Ig-tailed fusion proteins are well known to those of skill in the art. See, for example, U.S. Pat. No. 5,116,964, International Publication No. WO 98/23289, International Publication No. WO 97/34631, and U.S. Pat. No. 6,277,375, each of which is incorporated herein by reference in its entirety. Further, in addition to the Ig-region encoded by the IgG1 vector, the Fc portion of the Ig region utilized can be modified, by amino acid substitutions, to reduce complement activation and Fc binding. (See, e.g., European Patent No. 239400 B1, Aug. 3, 1994; U.S. Pat. Nos. 5,763,416 and 5,962,427).

5.3. Methods of Expressing Viral Interferon Antagonists or Via Fusion Proteins

The viral interferon antagonists or VIA fusion proteins can be produced by any method known in the art for the synthesis of proteins, in particular, by chemical synthesis or preferably, by recombinant expression techniques.

The nucleotide sequence coding for a viral interferon antagonist, or a VIA fusion protein can be inserted into an appropriate expression vector, i.e., a vector which contains the necessary elements for the transcription and translation of the inserted protein-coding sequence. In one embodiment, the viral interferon antagonist is altered to decrease immunogenicity so as to prevent or diminish a host immune response against the viral interferon antagonist. In a preferred embodiment, the viral interferon antagonist is NS-1 of influenza A virus that is altered to decrease its immunogenicity. The necessary transcriptional and translational signals can also be supplied by the native gene encoding the viral interferon antagonist or its flanking regions. A variety of host-vector systems may be utilized to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Any of the methods well known to one of skill in the art for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of a nucleic acid sequence encoding a viral interferon antagonist, or a VIA fusion protein may be regulated by a second nucleic acid sequence so that the viral interferon antagonist, or VIA fusion protein is expressed in a host transformed with the recombinant DNA molecule. For example, expression of a viral interferon antagonist or VIA fusion protein may be controlled by any promoter or enhancer element known in the art, including constitutive, inducible and tissue-specific regulatory elements.

Promoters which may be used to control the expression of a nucleotide sequence encoding a viral interferon antagonist or VIA fusion protein include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, the expression of a viral interferon antagonist or a VIA fusion protein is controlled by a steroid promoter. In accordance with this embodiment, the expression of a viral interferon antagonist or VIA fusion protein is inducible by a steroid (e.g., RU486). Accordingly, the expression of a viral interferon antagonist or VIA fusion protein regulated by a steroid promoter can be induced in vitro or in vivo by contacting a cell engineered to contain the viral interferon antagonist or VIA fusion protein with a steroid.

In a specific embodiment, a vector is used that comprises a promoter operably linked to a viral interferon antagonist-encoding nucleic acid, one or more origins of replication, and, optionally, one or more selectable markers (e.g., an antibiotic resistance gene). In another embodiment, a vector is used that comprises a promoter operably linked to a VIA fusion protein-encoding nucleic acid, one or more origins of replication, and, optionally, one or more selectable markers (e.g., an antibiotic resistance gene).

In bacterial systems, a number of expression vectors may be advantageously selected depending upon the use intended for the viral interferon antagonist or VIA fusion protein being expressed. For example, when a large quantity of such a protein is to be produced, for the generation of pharmaceutical compositions, vectors which direct the expression of high levels of protein products that are readily purified may be desirable.

In mammalian host cells, a number of viral-based expression systems (e.g., retroviral vectors, adenoviral vectors, and adeno-associated viral vectors) may be utilized to express a viral interferon antagonist or VIA fusion protein. In one embodiment, a native virus naturally encoding a viral interferon antagonist is used to express the viral interferon antagonist in mammalian host cells. In an alterative embodiment, a virus heterologous to the virus from which a viral interferon antagonist is derived is used to express the viral interferon antagonist in mammalian host cells.

Attenuated viruses that express a viral interferon antagonist are included in one aspect of the invention. In one embodiment, an attenuated virus with interferon antagonist activity is used to deliver a viral interferon antagonist to a mammalian host cell. Attenuated viruses with interferon antagonist activity include naturally occurring attenuated mutants with interferon antagonist activity, mutagen-induced attenuated mutants with interferon antagonist activity, and genetically engineered attenuated viruses with interferon antagonist activity. In another embodiment, an attenuated virus lacking or with impaired interferon antagonist activity is used to deliver a viral interferon antagonist or VIA fusion protein to a mammalian host cell.

Any attenuated mutant virus can be selected and used in accordance with the invention to express a viral interferon antagonist. In one embodiment, naturally occurring attenuated mutants or variants, or spontaneous attenuated mutants are selected that have the ability to antagonize the cellular interferon response. In another embodiment, attenuated mutant viruses generated by exposing the virus to one or more mutagens, such as ultraviolet irradiation or chemical mutagens, or by multiple passages and/or passage in non-permissive hosts, are selected for those viruses that have the ability to antagonize the cellular interferon response. Screening in a differential growth system can be used to select for those mutants having an attenuated phenotype. For viruses with segmented genomes, the attenuated phenotype can be transferred to another strain having a viral interferon antagonist by reassortment (i.e., by coinfection of the attenuated virus and the desired strain, and selection for reassortants displaying both phenotypes). In a preferred embodiment, an attenuated virus is genetically engineered to express a heterologous viral interferon antagonist.

Mutations can be engineered into a negative strand RNA virus such as influenza, RSV, NDV, VSV and PIV, using “reverse genetics” approaches. Reverse genetics can also be used to engineer a heterologous viral interferon antagonist or VIA fusion protein into a virus. In this way, natural or other mutations which confer an attenuated phenotype and a viral interferon antagonist or VIA fusion can be engineered into any negative strand RNA virus. For example, deletions, insertions or substitutions of the coding region of the gene responsible for attenuation can be engineered. Preferably, the gene responsible for attenuation of the virus is not a viral interferon antagonist. Insertions or substitutions can be used to express the viral interferon antagonist or VIA fusion. Deletions, substitutions or insertions in the non-coding region of the gene responsible for attenuation are also contemplated. To this end, mutations in the signals responsible for the transcription, replication, polyadenylation and/or packaging of the gene responsible for the attenuation can be engineered. For example, in influenza, such modifications can include but are not limited to: substitution of the non-coding regions of an influenza A virus gene by the non-coding regions of an influenza B virus gene (Muster, et al., 1991, Proc. Natl. Acad. Sci. USA, 88:5177), base pairs exchanges in the non-coding regions of an influenza virus gene (Fodor, et al., 1998, J. Virol. 72:6283), mutations in the promoter region of an influenza virus gene (Piccone, et al., 1993, Virus Res. 28:99; Li, et al., 1992, J. Virol. 66:4331), substitutions and deletions in the stretch of uridine residues at the 5′ end of an influenza virus gene affecting polyadenylation (Luo, et al., 1991, J. Virol. 65:2861; Li, et al., J. Virol. 1994, 68(2):1245-9). Such mutations, for example, to the promoter, could down-regulate the expression of any gene responsible for wild type growth. Preferably, deletions, insertions, or mutations made in the non-coding region of a viral interferon antagonist are not responsible for the attenuation of the virus. Mutations in viral genes which may regulate the expression of the gene responsible for IFN antagonist activity are also within the scope of viruses that can be used in accordance with the invention.

The reverse genetics technique involves the preparation of synthetic recombinant viral RNAs that contain the non-coding regions of the negative strand virus RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in European Patent Publication EP 0702085A1, published Feb. 20, 1996; in U.S. Pat. No. 6,146,642; in International Patent Publications PCT WO97/12032 published Apr. 3, 1997; WO96/34625 published Nov. 7, 1996; in European Patent Publication EP-A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 47SA 1 published Jun. 25, 1997, each of which is incorporated by reference herein in its entirety. Reverse genetics can thus be used to express any viral interferon antagonist or VIA fusion in any negative strand RNA virus.

Reverse genetics techniques can also be used to engineer additional mutations to other viral genes important for targeting the viral interferon antagonist—i.e., the epitopes that confer binding to particular cell types or tissues. Thus, epitopes which alter the tropism of the virus in vivo can be engineered into attenuated viruses with interferon antagonist activity to direct the delivery of the viral interferon antagonist or VIA fusion to the cell type or tissue in need of the interferon antagonist.

The helper-free plasmid technology can also be utilized to engineer an RNA virus, preferably a heterologous RNA virus, to express a viral interferon antagonist or VIA fusion protein. For a description of helper-free plasmid technology see, e.g., International Publication No. WO 01/04333, which is incorporated herein by reference in its entirety.

In another embodiment, a DNA virus (e.g., vaccinia, adenovirus, baculovirus) or a positive strand RNA virus (e.g., polio virus) is used to express a viral interferon antagonist or VIA fusion protein. In accordance with this embodiment, the DNA virus or positive strand RNA virus may express a native viral interferon antagonist or be engineered to express a heterologous viral interferon antagonist or a VIA fusion protein. In such cases, recombinant DNA techniques which are well known in the art may be used to engineer the expression of a viral interferon antagonist or VIA fusion (e.g., see U.S. Pat. No. 4,769,330 to Paoletti, U.S. Pat. No. 4,215,051 to Smith each of which is incorporated herein by reference in its entirety). In a specific embodiment, the DNA virus or positive strand RNA virus used to express a viral interferon antagonist or VIA fusion protein has been attenuated by any technique well known to one of skill in the art. Preferably, the attenuated phenotype of the virus is not due to any mutations which impair the activity of a viral interferon antagonist.

In cases where an adenovirus is used as an expression vector, the viral interferon antagonist or VIA fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the interferon antagonist in infected hosts (e.g, see Logan & Shenk, 1984, Proc. Natl. Acad. Sci. USA 81:355-359). Specific initiation signals may also be required for efficient translation of the inserted interferon antagonist coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a viral interferon antagonist or VIA fusion protein can be identified by three general approaches: (a) nucleic acid hybridization, (b) presence or absence of “marker” gene functions, and (c) expression of inserted sequences. In the first approach, the presence of a gene encoding a viral interferon antagonist or VIA fusion protein inserted in an expression vector can be detected by nucleic acid hybridization using probes comprising sequences that are homologous to an inserted gene encoding a viral interferon antagonist or VIA fusion protein. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding a viral interferon antagonist or VIA fusion protein in the vector. For example, if the gene encoding the viral interferon antagonist is inserted within the marker gene sequence of the vector, recombinants containing the gene encoding the viral interferon antagonist insert can be identified by the absence of the marker gene function. In the third approach, recombinant expression vectors can be identified by assaying the gene product (i.e., a viral interferon antagonist or VIA fusion protein) expressed by the recombinant cell. Such assays can be based, for example, on the physical or functional properties of the viral interferon antagonist in in vitro assay systems, e.g., binding with an anti-viral interferon antagonist antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered viral interferon antagonist or VIA fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of a viral interferon antagonist or VIA fusion protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, W138 Host cells, cell lines or bacteria can be transformed or transfected with expression vectors containing nucleotide sequences encoding viral interferon antagonists or VIA fusion proteins using any method well known to one of skill in the art. For example, calcium phosphate precipitation, electroporation, liposomes can be used to transfect a cell or cell line. Cells or cell lines can be transiently or stably transfected with an expression vector.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a viral interferon antagonist or VIA fusion protein. Such engineered cell lines may be particularly useful in the production of large quantities of a viral interferon antagonist or VIA fusion protein. Further, such engineered cell lines may be useful in the screening and evaluation of compounds that modulate the expression and/or activity of a viral interferon antagonist or VIA fusion protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Proc. Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150: 1); and hygro, which confers resistance to hygromycin (Santerre, et al., 1984, Gene 30:147) genes.

5.4. Therapeutic/Prophylactic Utility of Viral Interferon Antagonists

The present invention encompasses the prophylactic and therapeutic use of viral interferon antagonists and/or VIA fusion protein. In one embodiment, the viral interferon antagonist or VIA fusion protein is not a vaccine. In a specific embodiment, the viral interferon antagonist or VIA fusion protein does not elicit an immune response directed to the viral interferon antagonist or VIA fusion protein. In another embodiment, the viral interferon antagonist or VIA fusion protein does not elicit an antibody response to a viral interferon antagonist or VIA fusion protein. In another embodiment, a first dose of an viral interferon antagonist or VIA fusion protein does not elicit an immune response in a subject which results in rejection of the viral interferon antagonist or VIA fusion protein when subsequent doses are administered to a subject.

The invention provides methods of modulating the immune response in a subject, said methods comprising administering to a subject in need thereof one or more viral interferon antagonists. In particular, the present invention provides methods of modulating the cellular interferon response in a subject, said methods comprising administering to subject in need thereof one or more viral interferon antagonists. Preferably, the administration of one or more viral interferon antagonists to a subject reduces or inhibits viral interferon expression and/or activity. The invention also provides methods of modulating Th1/Th2 differentiation and/or Th1 replication in a subject, said methods comprising administering to a subject in need thereof one or more viral interferon antagonists or VIA fusion proteins. Preferably, the administration of one or more viral interferon antagonists to a subject reduces or inhibits the differentiation of Th0 cells to Th1 cells and/or reduces or inhibits Th1 replication.

The invention also provides methods of preventing, treating, or ameliorating one or more disorders characterized by aberrant interferon expression and/or activity, said methods comprising administering to a subject in need thereof a prophylactically or therapeutically effective amount of one or more viral interferon antagonists or VIA fusion proteins. In a specific embodiment, the invention provides methods of preventing, treating, or ameliorating one or more symptoms associated with a Th1 or Th1-like related disorder in a subject, said methods comprising administering to said subject a prophylactically or therapeutically effective amount of one or more viral interferon antagonists or VIA fusion proteins. Examples of Th-1 or Th-1-like related disorders include, but are not limited to, chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease and sarcoidosis.

In a preferred embodiment, the invention provides methods of preventing, treating or ameliorating one or more symptoms associated with an inflammatory disorder, said methods comprising administering to a subject in need thereof a prophylactically or therapeutically effective amount of one or more viral interferon antagonists or VIA fusion proteins. Examples of inflammatory disorders include, but are not limited to, arthritis (e.g., rheumatoid arthritis), psoriasis, multiple sclerosis, inflammatory bowel syndrome, fibrosis, lupus, and Crohn's disease.

The present invention encompasses the use of viral interferon antagonists and/or VIA fusion proteins to enhance gene expression. In particular, the invention provides methods of enhancing the translation of endogenous genes or foreign genes that have been introduced into a cell by gene therapy, said methods comprising contacting a cell with one or more viral interferon antagonists or VIA fusion proteins. The invention also provides methods of enhancing the expression of an endogenous gene or foreign gene introduced by gene therapy in a subject, said methods comprising administering to a subject in need thereof one or more viral interferon or VIA fusion proteins. Examples of subjects which may need enhanced expression of an endogenous gene or foreign gene include, but are not limited to, individuals with cystic fibrosis, diabetes, hemophelia or sickle cell disease.

In certain embodiments, in accordance with the methods of the invention one or more viral interferon antagonists or VIA fusion proteins are administered to a subject as polypeptides. In certain other embodiments, in accordance with the methods of the invention nucleic acids encoding one or more viral interferon antagonists or VIA are administered to a subject. Any viral protein, polypeptide, fragment, derivative or analog thereof with interferon antagonist activity can be used in accordance with the methods of the present invention. Examples of viral interferon antagonists that can be used in accordance with the methods of the invention include, but are not limited to, VP35 of Ebola virus, NS2 of RSV, E3L of vaccina virus, VA RNA₁ of adenovirus and NS1 of influenza virus. In certain embodiments, the viral interferon antagonist used in accordance with the methods of the invention is influenza virus NS1. In certain other embodiments, the viral interferon antagonist used in accordance with the methods of the invention is not influenza virus NS1.

5.5. Compositions and Methods of Administering Viral Interferon Antagonists & Via Fusion Proteins

The present invention provides compositions comprising one or more nucleotide sequences encoding one or more viral interferon antagonists, and a carrier. The present invention also provides compositions comprising one or more viral interferon antagonists, and a carrier. The present invention also provides compositions comprising one or more nucleotide sequences encoding one or more VIA fusion proteins, and a carrier. The present invention also provides compositions comprising one or more VIA fusion proteins, and a carrier. The present invention also provides compositions comprising one or more viral interferon antagonists and one or more VIA fusion proteins, and a carrier. The present invention further provides compositions comprising one or more nucleotide sequences encoding one or more viral interferon antagonists and one or more nucleotide sequences encoding one or more VIA fusion proteins, and a carrier.

The present invention provides pharmaceutical compositions comprising one or more nucleotide sequences encoding one or more viral interferon antagonists, and a pharmaceutically acceptable carrier. The present invention also provides pharmaceutical compositions comprising one or more viral interferon antagonists, and a pharmaceutically acceptable carrier. The present invention also provides pharmaceutical compositions comprising one or more nucleotide sequences encoding one or more VIA fusion proteins, and a pharmaceutically acceptable carrier. The present invention also provides pharmaceutical compositions comprising one or more VIA fusion proteins, and a pharmaceutically acceptable carrier. The present invention also provides pharmaceutical compositions comprising one or more viral interferon antagonists and one or more VIA fusion proteins, and a pharmaceutically acceptable carrier. The present invention further provides pharmaceutical compositions comprising one or more nucleotide sequences encoding one or more viral interferon antagonists and one or more nucleotide sequences encoding one or more VIA fusion proteins, and a pharmaceutically acceptable carrier. In a preferred embodiment, the pharmaceutical compositions are sterile and in suitable form for administration to a subject, preferably an animal subject, more preferably a mammalian subject, and most preferably a human subject.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of one or more viral interferon antagonists, or one or more VIA fusion proteins, in purified form, together with a suitable amount of carrier so as to provide the form for proper administration to the patient. The formulation should suit the mode of administration.

A pharmaceutical composition of the invention is formulated to be compatible with its intended route of administration. Examples of routes of administration include parenteral, e.g., intravenous, intradermal, subcutaneous, oral (e.g., inhalation), intranasal, transdermal (topical), transmucosal, and rectal administration.

The compositions of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

The pharmaceutical compositions may also be delivered in a controlled release or sustained release system. In one embodiment, a pump may be used (see Langer, supra; Sefton, CRC Crit. Ref. Biomed. Eng. 14:201 (1987); Buchwald et al., 1980, Surgery 88:507; and Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., 1985, Science 228:190; During et al., 1989, Ann. Neurol. 25:351; and Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533) and may be used in connection with the administration of compositions of the invention.

The compositions of the invention are advantageously used in methods to: modulate interferon expression and/or activity in vivo and/or in vitro; enhance gene expression; modulate Th1/Th2 differentiation and/or Th1 replication in vivo and/or in vitro; prevent, treat, or ameliorate one or more symptoms associated with an immune disorder characterized by aberrant IFN expression and/or activity; prevent, treat, or ameliorate one or more symptoms associated with a Th1 or Th1-like related disorder; or prevent, treat, or ameliorate one or more symptoms associated with an inflammatory disorder. Preferably, pharmaceutical compositions are used in the methods of the invention.

The amount of the pharmaceutical composition of the invention which will be effective in the treatment, prevention or amelioration of one or more symptoms of a disorder will depend on the nature of the disorder, and can be determined by standard clinical techniques. In addition, in vitro assays may optionally be employed to help identify optimal dosage ranges. The precise dose to be employed in the formulation will also depend on the route of administration, and the seriousness of the inflammatory disorder or aberrant interferon gene expression, and should be decided according to the judgment of the practitioner and each patient's circumstances. Effective doses may be extrapolated from dose-response curves derived from in vitro or animal model test systems. As defined herein, a prophylactically or therapeutically effective amount of protein or polypeptide (i.e., an effective dosage) ranges from about 0.001 to 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. Effective doses of a viral interferon antagonist or VIA fusion protein may be administered on more than one occasion to a subject in need thereof. In one embodiment, the same viral interferon antagonist or VIA fusion protein is administered to a subject in need thereof on more than one occasion. In another embodiment, a viral interferon antagonist or VIA fusion protein different from the viral interferon antagonist or VIA fusion protein administered to the subject on previous occasions is administered to the subject as needed.

The nucleotide sequences encoding viral interferon antagonists can be inserted into vectors and such vectors can used as gene therapy vectors for ex vivo and in vivo gene therapy. The nucleotide sequences encoding VIA fusion proteins can also be inserted into vectors and such vectors can used as gene therapy vectors. Any gene therapy vector known to one of skill in the art can be used to deliver nucleotide sequences. For example, adenovirus, adeno-associated virus, influenza virus and retrovirus vectors well-known to one of skill in the art can be used to deliver nucleotide sequences.

Gene therapy vectors can be delivered to an animal by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a matrix with in situ scaffolding in which the gene delivery vehicle is contained (see, e.g., European Patent No. EP 0 741 785 B1 and U.S. Pat. No. 5,962,427). Alternatively, where the complete gene delivery vector can be produced intact from recombinant cells, the pharmaceutical preparation can include one or more cells which produce the gene delivery system. In one embodiment, a viral vector heterologous to the virus from which a viral interferon antagonist is derived is used as a gene therapy vector for the delivery of the viral interferon antagonist. In a preferred embodiment, an attenuated viral vector heterologous to the virus from which a viral interferon antagonist is derived is used as a gene therapy vector for the delivery of the viral interferon antagonist. In certain embodiments of the invention, viral vectors are not used for methods of delivering viral interferon antagonists or VIA fusion proteins to a subject.

5.6. Combinatorial Therapy

The invention encompasses the use of one or more viral interferon antagonists or VIA fusion proteins in combinatorial therapies for the prevention, treatment or amelioration of one or more symptoms associated with an immune disorder. In particular, the present invention provides methods for preventing, treating or ameliorating one or more symptoms associated with an immune disorder characterized by aberrant IFN expression and/or activity in a subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder. The present invention also provides methods for preventing, treating or ameliorating one or more symptoms associated with a Th1 or Th1-like related disorder in subject, said methods comprising administering to said subject one or more viral interferon antagonists or VIA fusion proteins prior to, subsequent to, or concomitantly with the administration of one or more known therapies for preventing, treating or ameliorating one or more symptoms of such a disorder.

In a preferred embodiment, the present invention provides methods of preventing, treating or ameliorating one or more symptoms of an inflammatory disorder in subject, said methods comprising administering to said subject one or more viral interferon antagonists or VIA fusion proteins prior to, subsequent to, or concomitantly with the administration of one or more other anti-inflammatory agents for preventing, treating or ameliorating one or more symptoms of such a disorder. Examples of anti-inflammatory agents include, but are not limited to, aspirin, leflunomide (Arava), non-steroidal anti-inflammatory agents (e.g., ibuprofen, fenoprofen, indomethacin, and naproxen), and anti-TNFα agents (e.g., infliximab (Remicade) and etanercept (Enbrel)). One or more viral interferon antagonists may also be advantageously utilized in combination with one or more immunomodulatory agents (such as, e.g., Cyclosporin A (CsA), methylprednisolone (MP), corticosteroids, OKT3 (anti-CD3 monoclonal human antibody), mycophenolate mofetil, rapamycin (sirolimus), mizoribine, deoxyspergualin, macrolide antibiotics (e.g., FK506 (tacrolimus), brequinar, and malononitriloamindes.(e.g., leflunamide)), and anti-IL-2R antibodies (e.g., anti-Tac monoclonal antibody and BT 536)), or with lymphokines or hematopoietic growth factors (e.g., IL-10), or with anti-angiogenic factors (e.g., angiostatin, an antagonist of Integrin αᵥβ₃ (e.g., VITAXIN™), a TNFα antagonist (e.g., anti-TNFα antibody), or endostatin) for the prevention, treatment or amelioration of one or more symptoms associated with an inflammatory disorder.

In a specific embodiment, one or more viral interferon antagonists or VIA fusion proteins are administered prior to (e.g., 2 hours, 6 hours, 12 hours, 1 day, 4 days, 6 days, 12 days, 14 days, 1 month or several months before) the administration of one or more anti-inflammatory agents. In another specific embodiment, one or more viral interferon antagonists or VIA fusion proteins are administered subsequent to (e.g., 2 hours, 6 hours, 12 hours, I day, 4 days, 6 days, 12 days, 14 days, 1 month or several months after) the administration of one or more anti-inflammatory agents. In a specific embodiment, one or more viral interferon antagonists or VIA fusion proteins are administered concomitantly with one or more anti-inflammatory agents.

In another specific embodiment, one or more viral interferon antagonists or VIA fusion proteins are utilized in combination with one or more known therapeutic or prophylactic agents for a particular inflammatory disorder. For example, one or more viral interferon antagonists may be utilized in combination with one or more corticosteroids and/or one or more nonsteroidal anti-inflammatory agents to prevent, treat, or ameliorate one or more symptoms of systemic lupus erythematosus. In another example, one or more viral interferon antagonists may be utilized in combination with aspirin, leflunomide (Arava), one or more non-steroidal anti-inflammatory agents (e.g., ibuprofen, fenoprofen, indomethacin, and naproxen), and/or one or more anti-TNFα agents (e.g., infliximab (Remicade) and etanercept (Enbrel)) to prevent, treat or ameliorate one or more symptoms of rheumatoid arthritis.

The invention encompasses the use of the pharmaceutical compositions of the invention in combinatorial therapies for the prevention, treatment, and amelioration of one or more symptoms of an immune disorder characterized by aberrant interferon expression and/or activity. The pharmaceutical compositions of the invention can be administered prior to, subsequent to, or concomitantly with the administration of any other prophylactic or therapeutic composition for the prevention, treatment or amelioration of a Th1 or Th1-like related disorder. The pharmaceutical compositions of the invention can also be administered prior to, subsequent to, or concomitantly with the administration of any other prophylactic or therapeutic composition for the prevention, treatment or amelioration of an inflammatory disorder.

The present invention encompasses the use of one or more viral interferon antagonists or VIA fusion proteins in cycling therapy for the treatment, prevention, or amelioration of one or more symptoms of an immune disorder characterized by aberrant interferon expression and/or activity, a Th1 or Th1-like disorder, or an inflammatory disorder. The invention also encompasses combinations of one or more viral interferon antagonists or VIA fusion proteins and one or more therapeutic agents known in the art that have different sites of action. Such a combination provides an improved therapy based on the dual action of these therapeutics whether the combination is synergistic or additive. Preferably, the combinatorial therapies of the present invention have an additive or synergistic effect while reducing or avoiding unwanted or adverse side effects.

5.7. Methods for Assessing the Prophylactic/Therapeutic Utility of Viral Interferon Antagonists

Viral interferon antagonists, VIA fusion proteins, and the compositions of the invention are, preferably, tested in vitro, in a cell culture system, and in an animal model organism, such as a mouse, for toxicity and the desired prophylactic or therapeutic utility prior to use in humans. In vitro assays can be performed in any cell line or cells from any patient tissue sample. For example, assays which can be used to determine whether administration of a specific composition is indicated, include cell culture assays in which a patient tissue sample is grown in culture, and exposed to or otherwise contacted with a composition, and the effect of such composition upon the tissue sample is observed. The tissue sample can be obtained by biopsy from the patient.

Viral interferon antagonists, VIA fusion proteins and compositions of the invention can be assessed for their ability to modulate cytokine expression, in particular their ability to inhibit or reduce interferon expression using assays well known in the art or described herein. Cytokine expression can be assayed, for example, by immunoassays including, but are not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. Viral interferon antagonists, VIA fusion proteins and compositions of the invention can also be assessed for their ability to inhibit or reduce interferon activity using assays well known in the art or described herein. For example, the activation of signaling molecules such as JAKS, IRF3, and STATS can be assayed, for example, by kinase assays and electromobility shift assays (EMSAs). Further, the expression of genes known to be induced by interferon (e.g., Mx, PKR, 2,5-oligoadenylatesynthetase, and major histocompatibility complex (MHC) type I) can be analyzed by techniques known to one of skill in the art such as, e.g., northern blot analysis, PCR, and immunoassays). Alternatively, test cells such as human embryonic kidney cells or human osteogenic sarcoma cells, are engineered to transiently or constitutively express reporter genes such as luciferase or chloramphenicol transferase (CAT) reporter gene under the control of an interferon stimulated response element, such as the interferon-stimulated prooter of the ISG-54K gene (Bluyssen et al., 1994, Eur. J. Biochem. 220:395-402). Such cell lines are assessed for reporter gene expression in the presence and absence of a viral interferon antagonist, VIA fusion protein or composition of the invention by techniques well known to one of skill in the art or described herein (e.g., by northern blot analysis, PCR, or immunoassay). The ability of a viral interferon antagonist, VIA fusion protein or composition of the invention to modulate Th1/Th2 differentiation can be assessed by, e.g., analyzing cytokine profiles using techniques well known to one of skill in the art. Further, the ability of a viral interferon antagonist, VIA fusion protein or composition of the invention to modulate Th1 replication can be assessed by T cell proliferation assays well known to one of skill in the art, including, e.g., FACS and trypan blue cell counts. Viral interferon antagonists, VIA fusion proteins and compositions of the invention can also be tested in suitable animal model systems prior to use in humans. Such animal model systems include but are not limited to rats, mice, chicken, cows, monkeys, pigs, dogs, rabbits, etc. For in vivo testing, prior to administration to humans, any animal model system known in the art may be used. For example, a collagen-induced arthritis (CIA) model or other animal models for arthritis can be utilized to determine the efficacy of the compositions of the invention (see, e.g., Holmdahl, R., 1999, Curr. Biol. 15:R528-530; and Crofford L. J. and Wilder R. L., “Arthritis and Autoimmunity in Animals”, in Arthritis and Allied Conditions: A Textbook of Rheumatology, McCarty et al. (eds), Chapter 30 (Lee and Febiger, 1993)).

The principal animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), incorporated herein by reference in its entirety. CIA can be induced by the administration of heterologous type II collagen and is an animal model for rheumatoid arthritis (RA) (Trenthorn et al., 1977, J. Exp. Med.146:857; and Courtenay et al., 1980, Nature 283:665; and Cathcart et at, 1986, Lab. Invest. 54:26). Carrageenan-induced arthritis has been used in rat, rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art. It is apparent to the skilled artisan that, based upon the present disclosure, transgenic animals can be produced with “knock-out” mutations of the gene or genes encoding any cellular function required for development of symptoms associated with a Th1 or Th1-like related disorder or an inflammatory disorder.

The anti-inflammatory activity of a viral interferon antagonist, VIA fusion protein or composition of the invention can also be determined by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for anti-inflammatory activity of most NSAIDs, and is considered predictive of human efficacy. The anti-inflammatory activity of the test therapies is expressed as the percent inhibition of the increase in hind paw weight of the test group relative to the vehicle dosed control group.

The toxicity of a composition of the invention can be assessed using standard cell culture or experimental animal procedures well known to one of skill in the art including, e.g. the determination of the LD₅₀ (the dose lethal to 50% of the population) and the ED₅₀ (the dose therapeutically effective in 50% of the population). The dose ratio between toxic and therpeutic effects is the therapeutic index and it can be expressed as the ratio of LD₅₀/ED₅₀. Compositions of the invention that exhibit large therapeutic indices are preferred. While compositions that exhibit toxic side effects may be used, care should be taken to delivery the composition in a delivery system that targets the composition to the site of the affected tissue in order to minimize potential damage to unaffected cells, thereby reducing side effects.

The data obtained from the cell culture assays and animal studies can be used in formulating a range of dosage of a viral interferon antagonist or VIA fusion protein for use in humans. The dosage of such agents lies preferably within a range of circulating concentration that include the ED₅₀ with little or no toxicity. The dosage may vary within this range depending upon the dosage form employed and the route of administration utilized. For a viral interferon antagonist or VIA fusion protein used in the methods of the invention, the prophylactically or therapeutically effective dose can be estimated initially from cell culture assays. A dose may be formulated in animal models to achieve a circulating plasma concentration that includes the IC₅₀ (i.e., the concentration of the viral interferon antagonist or VIA fusion protein that achieves a half-maximal inhibition of symptoms) as determined in cell culture. Such information can be used to more accurately determine the useful doses in humans.

Further, any assays known to those skilled in the art can be used to evaluate the prophylactic and/or therapeutic utility of the combinatorial therapies disclosed herein for inflammatory diseases.

5.8. Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., the viral interferon antagonist, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, indicated interferon production levels, indicated Th-1/Th-2 lymphocyte counts, indicated total T cell counts and other monitoring information.

More specifically, the invention further provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a viral interferon antagonist or VIA fusion protein, and wherein said packaging material includes instruction means which indicate that said viral interferon antagonist or VIA fusion protein can be used to treat, prevent or ameliorate one or more symptoms of an immune disorder characterized by aberrant interferon expression and/or activity, a Th1 or Th-like related disorder, or an inflammatory disorder by administering specific doses and using specific dosing regimens as described herein in order to achieve the reduced or modulated interferon response as described herein. In a preferred embodiment, the instruction means indicate or suggest that interferon expression levels be monitored one or more times before and/or after a dose. For example, the instruction means can indicate that interferon expression levels be taken before the first dose and after one or more subsequent doses. In a specific embodiment, the instruction means indicate that the viral interferon antagonist is to be used to treat an inflammatory disorder. Suitable instruction means include printed labels, printed package inserts, tags, cassette tapes, and the like.

In specific embodiment, an article of manufacture comprises packaging material and an injectable form of a pharmaceutical agent contained within said packaging material, wherein said pharmaceutical agent comprises a viral interferon antagonist or VIA fusion protein and a pharmaceutically acceptable carrier, wherein said article of manufacture includes instruction means indicating a dosing regimen comprising administering an initial dosing, and optionally administering a subsequent dose or doses, of said pharmaceutical agent to a patient suffering from one or more symptoms associated with an inflammatory disorder, wherein the instruction means suggests a dosing regimen comprising an initial dosing that results in the viral interferon antagonist or VIA fusion protein modulating or decreasing the level of interferon gene expression.

The present invention provides that the adverse effects that may be reduced or avoided by the methods of the invention are indicated in informational material enclosed in an article of manufacture for use in preventing, treating or ameliorating one or more symptoms of an inflammatory disorder. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

The following series of examples are presented by way of illustration and not by way of limitation on the scope of the invention.

### 6. EXAMPLE

### Ebola Virus VP35 and Influenza A Virus NS1 Block Expression of CAT Gene Under the Control of an Interferon Sensitive Response Element

This example demonstrates that Ebola virus VP35 has interferon antagonist activity.

Expression of the Ebola VP35 Protein Blocks Induction of an ISRE Promoter

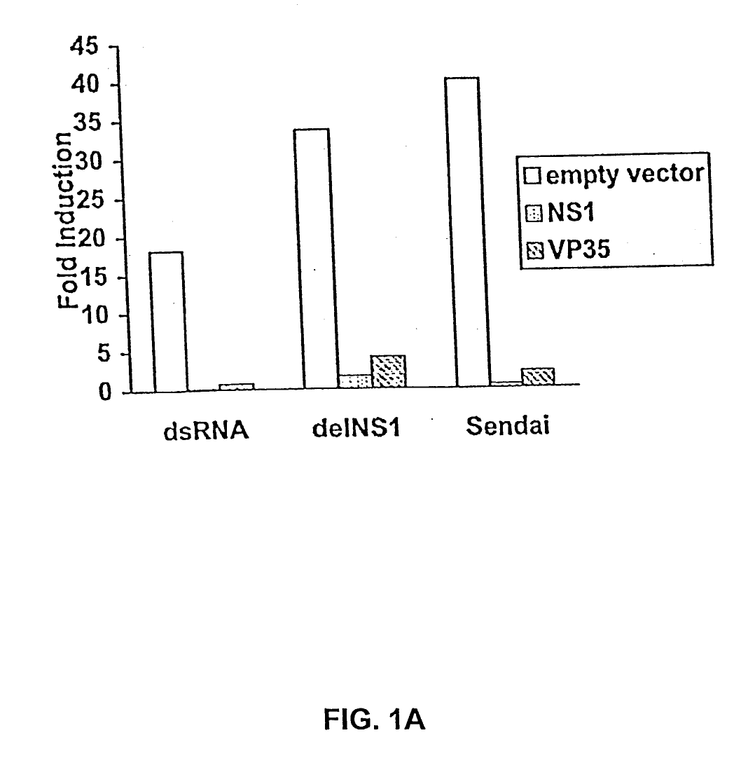

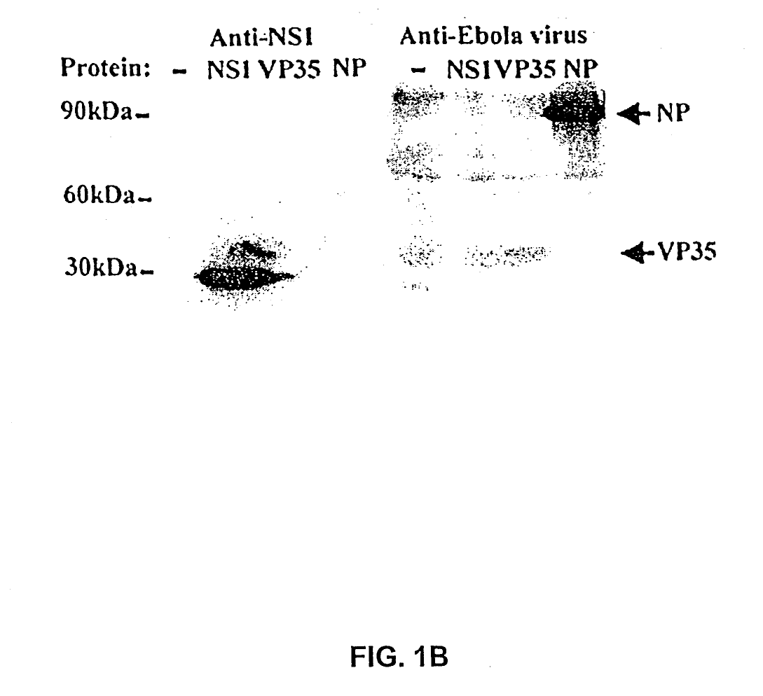

To determine whether VP35 inhibits the dsRNA- and virus-mediated activation of IFN-sensitive gene expression, cells were transfected with an ISRE-driven CAT-reporter plasmid and a constitutively expressed, simian virus 40 promoter-driven luciferase reporter plasmid. Additionally, the cells were transfected with empty vector, NS1 expression plasmid, VP35 expression plasmid, or, as an additional control, an Ebola virus NP expression plasmid. One day later, the cells were mock-treated, transfected with dsRNA, or infected with either influenza delNS 1 virus or with Sendai virus, strain Cantell (an attenuated strain known to induce large amounts of IFN). After an additional twenty four hours, cell lysates were prepared and assayed for CAT activity and luciferase activity (FIG. 1). Transfection of cells with dsRNA or infection with either influenza delNS1 virus or Sendai virus gave a strong induction of the IFN-sensitive promoter. When either NS1 or VP35 was present, expression from the IFN-responsive promoter was almost completely blocked. Levels of ISRE induction, normalized to levels of luciferase activity, are shown in FIG. 1A. Expression of the control luciferase reporter plasmid was not inhibited by expression of either NS1 or VP35. Expression of the Ebola virus NP, which did not complement growth of influenza delNS1 virus, did not inhibit activation of the ISRE promoter. Expression of the NS1, VP35, and NP proteins was confirmed by Western blotting (FIG. 1B). These results show that both NS1 and VP35 can block type I IFN production and/or signaling in response to either dsRNA treatment or to viral infection.

Expression of the Ebola Virus VP35 Protein Blocks Activation of the INF-β Promoter

In wild-type influenza A virus-infected cells, the NS1 protein blocks induction of type I IFN. This block is due, in large part, to the ability of NS1 to prevent activation of IRF-3 and NF-β, two transcription factors that play a critical role in stimulating the synthesis of IFN-β. Synthesis of IFN-β, in turn, plays an important role in the initiation of the type I IFN cascade (Marie et al. 1998 EMBO J. 17:6660-69). The Ebola virus VP35, therefore, was tested for its ability to block activation of the IFN-β promoter.

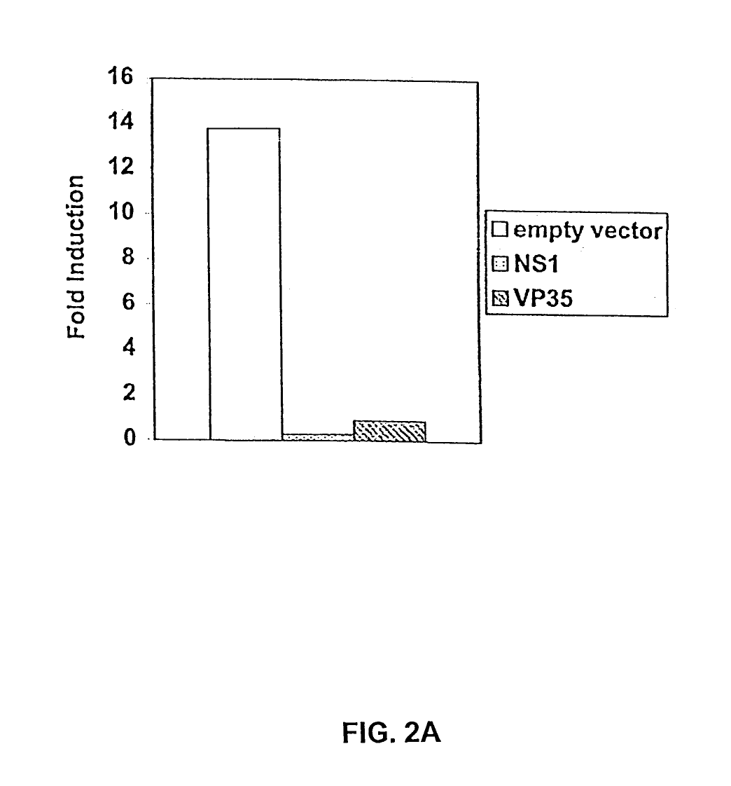

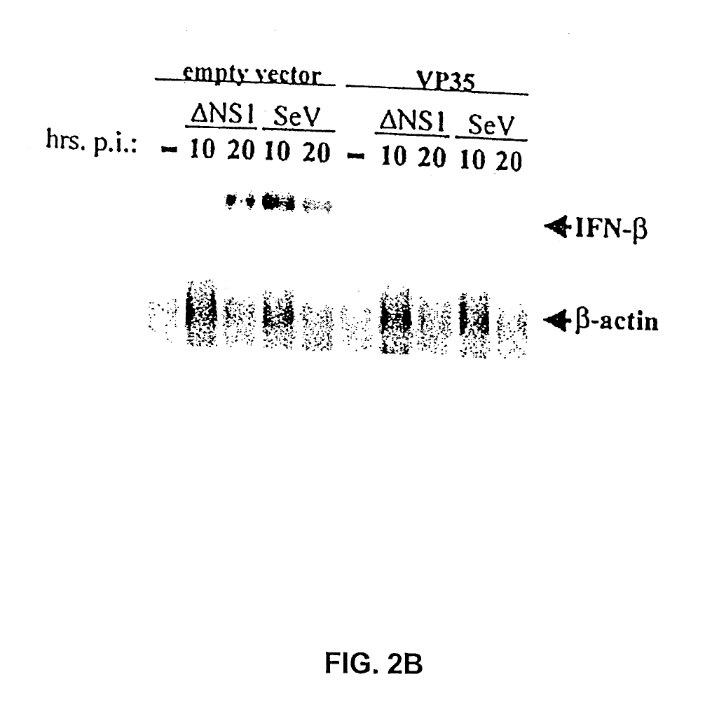

Empty vector, NS1 expression plasmid, or VP35 expression plasmid was cotransfected with a mouse IFN-β promoter-driven CAT reporter and a simian virus 40 promoter-driven luciferase reporter. When cells subsequently were transfected with dsRNA, a strong induction of the IFN-β promoter was observed in empty vector-transfected cells, but this induction was blocked when either NS1 or VP35 was expressed (FIG. 2A). It also was determined whether VP35 could block activation of the endogenous human IFN-β promoter. Cells were transfected with empty vector or VP35 expression plasmid and, twenty four hours later, mock-infected or infected with influenza delNS1 virus or with Sendai virus. Ten or twenty hours postinfection, total cellular RNA was isolated, and a Northern blot was performed to detect IFN-mRNA (FIG. 2B). Expression of VP35 clearly blocked induction of the endogenous IFN-β promoter. Before infection with either virus, IFN-β mRNA was undetectable. After infection, when the IFN-β mRNA levels were normalized to β-actin mRNA levels, it was found that, in influenza delNS 1 virus-infected cells, the presence of VP35 reduced IFN-β induction 8-fold at ten hours postinfection and 8.4-fold at twenty hours posttransfection. In Sendai virus-infected cells, the presence of VP35 reduced IFN-induction 6.1-fold at ten hours posttransfection and 5.9-fold at twenty hours posttransfection.

The Ebola Virus VP35 Blocks IFN Induction When Coexpressed with the Ebola Virus NP

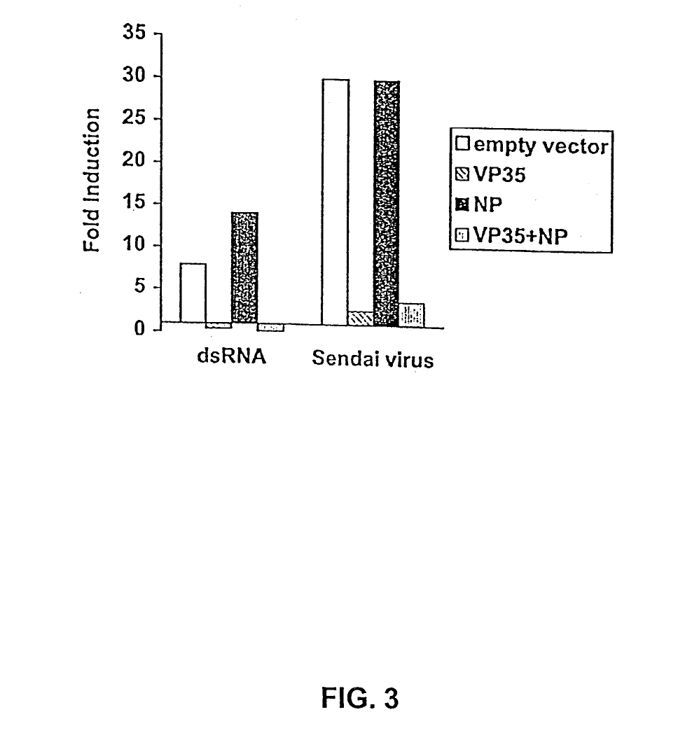

The VP35 protein is an essential component of the Ebola virus RNA synthesis complex and likely associates with the viral NP (Muhlberger et al. 1999 J. Virol. 73:2333-42; Becker et al. 1998 Virology 249:406-17). Therefore, it was determined whether Ebola virus VP35 retained its IFN-antagonizing properties when it was coexpressed with the Ebola virus NP. An ISRE-reporter assay was performed in which cells received either empty vector, VP35 alone, NP alone, or a combination of VP35 and NP. Twenty-four hours posttransfection, the cells were transfected with dsRNA or infected with Sendai virus. As seen previously, transfection with empty plasmid or with NP expression plasmid did not block activation of the ISRE promoter, but expression of VP35 did block its activation (FIG. 3). Further, coexpression of VP35 and NP was able to block ISRE activation to the same extent as expression of VP35 alone (FIG. 3). These data indicate that VP35, even when coexpressed with the Ebola virus NP, can act as an IFN antagonist.

The Ebola virus VP35 protein inhibits type I IFN induction when coexpressed with Ebola virus NP (FIG. 3). Fold induction of the IFN-inducible ISRE-driven reporter in the presence of empty vector, VP35, NP, or VP35 plus NP. 293 cells were transfected with a total of 4 μg of expression plasmid, including 2 μg of a plasmid encoding an individual protein and 2 μg of a second plasmid (either empty vector or a second expression plasmid) plus 0.3 μg each of the reporter plasmids pHISG-54-CAT and pGL2-Control. Twenty-four hours posttransfection, the cells were mock-treated or treated with the indicated IFN inducer. Twenty-four hours postinduction, CAT and luciferase assays were performed. The CAT activities were normalized to the corresponding luciferase activities to determine fold induction.

The production of an IFN antagonist contributes to the virulence of Ebola viruses. In humans, it appears that an appropriate cytokine response is related to the development of asymptomatic or nonfatal Ebola virus infection. Thus, a viral factor that influences type I IFN production influences viral pathology.

### 7. EXAMPLE

### NS1 Enhances Translation of mRNAS

The following example demonstrates the ability of a viral interferon antagonist to enhance the translation of mRNAs.

The influenza A virus NS1 protein has been reported to enhance translation of mRNAs (de la Luna et al. 1995 J. Virol. 67(4):2427-33; Enami et al. 1994 J. Virol. 68(3):1432-37). This ability is likely related to its ability to inhibit activation of the interferon-induced dsRNA-activated protein kinase, PKR (Hatada et al. 1999 J. Virol. 73(3):2425-33). However, it is not clear whether NS 1 inhibits PKR by sequestering dsRNA (Lu et al. 1999 Virology 214(1):222-28), by interacting directly with PKR (Tan et al. 1998 J. Interferon Cytokine Res. 18(9):757-66) or by a combination of the two mechanisms. The ability to enhance translation is a property characteristic of several viral-encoded PKR inhibitors, including adenovirus VA RNA₁ (Svensson et al. 1985 EMBO J.4(4):957-64) the vaccinia virus E3L protein (Davies et al. 1993 J. Virol. 67(3):1688-92), and perhaps the hepatitis C virus NS5A protein (Gale et al. 1997 Virology 230(2):217-27). These proteins also appear to confer interferon-resistance to the viruses (Beattie et al., 1995 J. Virol 69(1):499-505; Kitajewski et al. 1986 Cell 45(2):195-200).

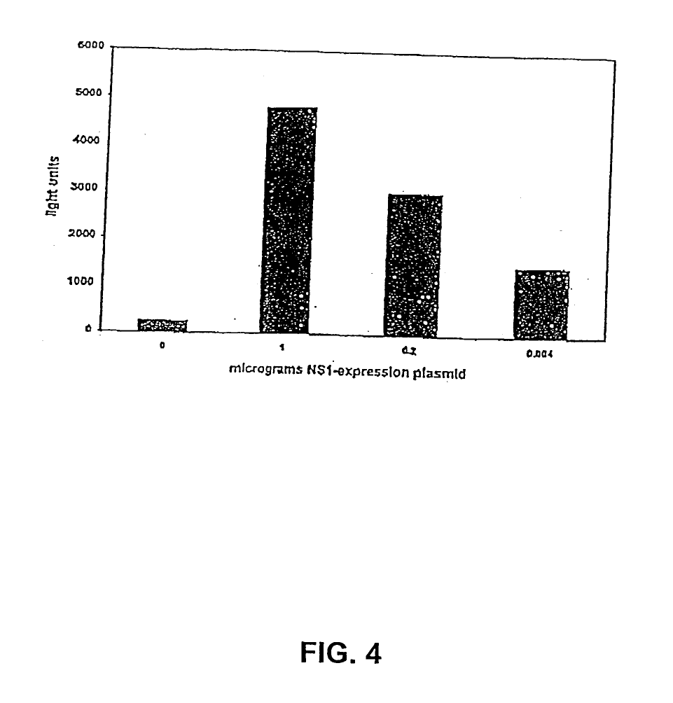

Therefore, the ability of the PR8 NS1 expression plasmid to enhance expression from a co-transfected reporter plasmid was tested. 293T cells were transfected with a total of 6 μg DNA. The 6 μg consisted of 4 μg pGL2-Control (Promega Corp.) (an SV40-promoter-driven, constitutively expressed luciferase reporter plasmid), 1 μg pEGFP-c1 (Clonetech Laboratories) (a CMV-promoter-driven green fluorescence protein (GFP) expression plasmid) and a combination of pCAGGS and pCAGGS-PR8 NS1 SAM totaling 1 μg. Transfections were performed containing 0, 1, 0.2 and 0.04 μg NS1 expression plasmid. Forty eight hours post-transfection, the cells were observed for GFP expression to confirm that dishes were transfected at comparable levels, and luciferase assays were performed. NS1-expression plasmid gave a 19.8-fold maximal stimulation of luciferase expression, and the enhancement was dose-dependent (FIG. 4).

### 8. EXAMPLE

### Expression in MDCK Cells of the Respiratory Syncytial Virus (RSV) NS2 Protein Complements Growth of delNS1 Virus

This example demonstrates that the human RSV NS2 protein has interferon antagonist activity To identify potential human RSV-encoded interferon antagonists, plasmids encoding human RSV proteins were screened for their ability to complement growth of the delNS1 virus on MDCK cells (Table 1). Expression of the human RSV NS2 protein in MDCK cells was found to stimulate growth of the mutant influenza virus. Therefore, the human RSV NS2 protein is likely to function as an interferon antagonist.

TABLE 1
Complementation of delNS1 virus growth by the human
RSV NS2 protein.

| Plasmid | Virus | HA Titer* |
|---|---|---|
| Empty vector | delNS1 | 0 |
| pcDNA3-PR8 NS1 SAM | delNS1 | 128 |
| pcDNA3-hRSV NS2 | delNS1 | 16 |

*Titer obtained by hemagglutination assay 48 hours post-infection.

The present invention is not to be limited in scope by the exemplified embodiments, which are intended as illustrations of single aspects of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety.

What is claimed is:

1. A pharmaceutical composition comprising one or more viral interferon antagonists in an amount effective to reduce or inhibit interferon expression or activity in vivo, and a pharmaceutically acceptable carrier.

2. The pharmaceutical composition of claim 1, wherein said viral interferon antagonist is influenza A virus NS1.

3. The pharmaceutical composition of claim 1, wherein said viral interferon antagonist is Ebola virus VP35.

4. The pharmaceutical composition of claim 1, wherein saidviral interferon antagonist is respiratory syncytial virus (RSV) NS2.

5. A method of modulating the interferon immune response in a subject, said method comprising administering to said subject an effective amount of one or more viral interferon antagonists, wherein said effective amount reduces or inhibits interferon expression.

6. A method of modulating Th1/Th2 differentiation or Th1 replication in a subject, said method comprising administering to said subject an effective amount of one or more viral interferon antagonists, wherein said effective amount reduces or inhibits interferon expression.

7. A method of treating, preventing or ameliorating one or more symptoms associated with a Th1-related immune disorder in a subject, said method comprising administering to a subject in need thereof a prophylactically or therapeutically effective amount of one or more viral interferon antagonists.

8. A method of treating, preventing or ameliorating one or more symptoms associated with an inflammatory disorder in a subject, said method comprising administering to a subject in need thereof a prophylactically or therapeutically effective amount of one or more viral interferon antagonists.

9. The method of claim 5, 6, 7, or 8, wherein said interferon antagonist is influenza A virus NS1.

10. The method of claim 5, 6, 7, or 8, wherein said interferon antagonist is RSV NS2.

11. The method of claim 5, 6, 7, or 8, wherein said interferon antagonist is Ebola virus VP35.

12. The method of claim 5, 6, 7, or 8, wherein the subject is a mammal.

13. The method of claim 12, wherein the mammal is a human.

14. The method of claim 7, wherein the Th1-related immune disorder is Crohn's disease, arthritis, Lyme disease, insulin-dependent diabetes, multiple sclerosis, Hashimoto's thyroiditis, Grave's disease, contact dermatitis, psoriasis, graft rejection, graft versus host disease, or sarcoidosis.
